SEQUENCE LISTING (4 sequences)

SEQ ID NO: 1 - DNA, 745 bp, Homo sapiens
  CDS (129)..(563)
gtatctgtggtaaacccagtgacacgggggagatgacatacaaaaagggcaggacctgagaaagattaagctgcaggctccctgcccataaaacagggtgtgaaaggcatctcagcggctgccccaccatggctacctgggccctcctgctccttgcagccatgctcctgggcaacccaggtctggtcttctctcgtctgagccctgagtactacgacctggcaagagcccacctgcgtgatgaggagaaatcctgcccgtgcctggcccaggagggcccccagggtgacctgttgaccaaaacacaggagctgggccgtgactacaggacctgtctgacgatagtccaaaaactgaagaagatggtggataagcccacccagagaagtgtttccaatgctgcgacccgggtgtgtaggacggggaggtcacgatggcgcgacgtctgcagaaatttcatgaggaggtatcagtctagagttacccagggcctcgtggccggagaaactgcccagcagatctgtgaggacctcaggttgtgtataccttctacaggtcccctctgagccctctcaccttgtcctgtggaagaagcacaggctcctgtcctcagatcccgggaacctcagcaacctctgccggctcctcgcttcctcgatccagaatccactctccagtctccctcccctgactccctctgctgtcctcccctctcacgagaataaagtgtcaagcaagaaaaaaa

SEQ ID NO: 2 - DNA, 24 bp, Homo sapiens
catctcagcggctgccccaccatg

SEQ ID NO: 3 - DNA, 27 bp, Homo sapiens
tgtataccttctacaggtcccctctga

SEQ ID NO: 4 - protein, 29 aa, Homo sapiens
RTGRSRWRDVCRNFMRRYQSRVTQGLVAG

(19) United States
(12) Patent Application Publication
(10) Pub. No.: US 2005/0239699 A1
(43) Pub. Date: Oct. 27, 2005

(54) Remedy for infections

(76) Inventors: Masaji Okada, Osaka (JP); Yasushi Takamori, Tokyo (JP); Kazuyuki Ogawa, Matoba Kawagoe-shi (JP); Kinya Nagata, Matoba Kawagoe-shi (JP)

Correspondence Address: DOUGLAS A. CHAIKIN;PENINSULA IP GROUP, A Professional Law Corporation, 26150 Bucks Run, Corral de Tierra, CA, 93908, US

(21) Appl. No.: 10/921,091

(22) Filed: Aug. 17, 2004

(30) Foreign Application Priority Data
Feb. 24, 2003 (JP) JP03/01970

(51) Int. Cl. A61K 38/17; A61K 48/00

(52) U.S. Cl. 514/12; 514/44

(57) ABSTRACT

According to the present invention, there is provided a remedy for infections containing 15K granulysin as an active ingredient, which has no perceived side effect, and is effective.

### TECHNICAL FIELD

The present invention is an invention relating to an infection remedy for treating infectious diseases such as tuberculosis.

### BACKGROUND OF THE INVENTION

In medical care, it goes without saying that the existence of a remedy for infectious diseases is indispensable. At present, a number of remedies for infections such as antibiotics and synthetic antibacterial agents are provided, and are used in medical care.

However, currently, an antibacterial agent which is mainly provided as a remedy for infectious diseases is in fact accompanied with an unavoidable problem of appearance of drug resistant bacteria. That is, sarcastic state continues in which by provision of a new antibacterial agent, a new drug resistant bacterium is produced.

For example, in tuberculosis occupying one of the highest number of patient with in single infectious disease, a tendency of recent increase becomes a worldwide problem. Further, the existence of a drug resistant bacterium having resistance to almost all antibiotics has been confirmed, and this problem is being revealed.

Recently, it has been revealed that a molecule called granulysin expressed in a Natural Killer cells (NK cell) or Cytotoxic T Lymphocytes

(CTL) has direct ability to kill and injure bacteria such as Mycobacterium tuberculosis [Stenger, S. et al., Science 282, 121-125(1998)].

Granulysin is produced as a precursor of 15K and, thereafter, processed into 9K in a cytotoxic granule, and it is known that this 9K granulysin has antibacterial activity (Pena, S. V. et al., J. Immunol, 158, 2680-2688 (1997)). Further, a pathway is reported in which perforin which is the molecule derived from the same cytotoxic intragranule perforates a target cell, granulysin enters into a cell therethrough, and kills and injures an infecting bacterium in a cell [Stenger, S. et. al., Science 282, 121-125 (1998)].

Like this, it is thought that granulysin plays an important role in defense for infection. It is hardly thought that active type 9K granulysin makes a bacterium thereto acquire resistance, and application to a remedy for infectious diseases having different characteristic from an antibiotic is contemplated. In addition, recently, the existence of perforin pathway regarding removal of an intracellular infectious bacterium by 9K granulysin is suspected (David, H. C. et al., J. Immunol, 167, 2734-2742 (2001)). Moreover, cytotoxity is perceived in this molecule (Pena, S. V. et al., J. Immunol, 158, 2680-2688 (1997)) and, when this is administered as such, considerable side effect toxicity is feared.

A problem to be solved by the invention is to study granulysin in further detail, and provide means for using this as a remedy for infectious diseases.

### DISCLOSURE OF THE INVENTION

In order to solve this problem, the present inventors intensively studied. As a result, the present inventors found out a novel pathway wherein 15K granulysin is incorporated into a macrophage and, thereafter, activated to kill and injure bacteria phagocytosed into a macrophage.

That is, the present inventors found out that use of 15K granulysin showing no cytotoxicity in itself as an active ingredient makes it possible to provide an efficacious remedy for infections which has little side effect and to which bacteria can acquire little tolerance.

The present invention is the invention providing an infection remedy of which active ingredient is 15K granulysin (hereinafter also referred to as the present remedy).

In the present invention, 15K granulysin is a protein, having a molecular weight of 15,000 (15k), consisting of 145 amino acids which is localized in cytotoxic granule as described above. In a cytotoxic granule, a part of 15K granulysin is cut, and 15K granulysin is present as a protein having a molecular weight of 9,000 (9k) (Pena, S. V., et al., J. Immunol., 158, 2680-2688 (1997), Stenger, S., et al., Science, 282, 121-125 (1998)).

### BRIEF DESCRIPTION OF THE DRAWINGS

FIG. 1 is a view showing results of study on antibacterial effect of 15K granulysin on Mycobacterium tuberculosis.

### DETAILED DESCRIPTION OF THE INVENTION

Embodiments of the present invention will be explained below.

A. Active Ingredient of Present Remedy

15K granulysin used as an active ingredient of the present remedy can is 5 be used by separating from a living body, but since 15K granulysin is a trace component in a living body, it is preferable to use as a recombinant protein obtained by expressing a gene encoding 15K granulysin.

Alternatively, it is also a suitable means to produce 15K granulysin in a living body, utilizing an intracorporeal expression vector for 15K granulysin in which a gene encoding 15K granulysin is inserted, as an active ingredient.

(i) Recombinant Protein of 15K Granulysin

A sequence of a gene encoding 15K granulysin has been already reported (Jongstra, et al., J. Exp. Med, 165, 601), specifically, a gene having a nucleotide sequence represented by SEQ ID NO: 1 and a 15K granulysin protein having an amino acid sequence corresponding thereto. Based on this, a gene encoding 15K granulysin is effectively prepared, and this gene is expressed, whereby, a recombinant protein of 15K granulysin can be prepared.

Specifically, a gene amplification product of a gene encoding 15K granulysin is prepared using nucleotide chains complementary to both ends of a sequence of a gene encoding 15K granulysin as an amplification primer, by a gene amplification method such as a PCR method.

This is transduced into a suitable gene expression vector, and desired 15K granulysin can be obtained from a suitable host such as Escherichia coli, Bacillus subtilis, yeast and insect cell transformed with such the recombinant vector.

It is preferable that, as a gene expression vector used herein, a vector usually harboring a promoter and an enhancer at a region upstream of a gene to be expressed, and a transcription termination sequence at a region downstream of the gene is used.

In addition, expression of a 15K granulysin gene is not limited to a direct expression system. For example, a fused protein expression system utilizing a D-galactosidase gene, a glutathione-S-transferase gene or a thioredoxin gene may be used.

As a gene expression vector using Escherichia coli as a host, there are exemplified pQE, pGEX, pT7-7, pMAL, pTrxFus, pET, and pNT26CII. As a vector using Bacillus subtilis as a host, there are exemplified pPL608, pNC3, pSM23, and pKH80.

In addition, as a vector using yeast as a host, there are exemplified pGT5, pDB248X, pART1, pREP1, YEp13, YRp7, and YCp50.

In addition, as a vector using a mammal cell or an insect cell as a host, there are exemplified p91023, pCDM8, pcDL.SRa296, pBCMGSNeo, pSV2dhfr, pSVdhfr, pAc373, pAcYM1, pRc/CMV, pREP4, and pcDNAI.

These gene expression vectors can be selected depending on the purpose of expression of 15K granulysin. For example, when 15K granulysin is expressed, it is preferable to select a gene expression vector for which Escherichia coli, Bacillus subtilis or yeast can be selected as a host. When 15K granulysin is expressed even at a small amount so that it has assuredly activity, it is preferable to select a gene expression vector for which a mammal cell or an insect cell can be selected as a host.

In addition, it is possible to select the existing gene expression vector as described above, but a gene expression vector is appropriately produced depending on a purpose, and this may be of course used.

Transfection of the gene expression vector in which a 15K granulysin gene is inserted, into a host cell, and a transformation method therewith can be performed by the general method, for example, a calcium chloride method and an electroporation method in the case of Escherichia coli and Bacillus subtilis as a host cell, and by the means such as a calcium phosphate method, an electroporation method and a liposome method in the case of a mammal cell and an insect cell as a host.

By the culture of the thus obtained transformant according to the conventional method, desired 15K granulysin is accumulated.

Medium used in such the culture can be appropriately selected depending on a host. For example, in the case of Escherichia coli as a host, LB medium and TB medium can be appropriately used and, in the case of mammal cells as a host, RPMI1640 medium can be appropriately used.

Isolation and purification of 15K granulysin from a culture obtained by this culture can be performed according to the conventional method. For example, isolation and purification can be performed by subjecting to the culture to various treating operations utilizing physical and/or chemical nature of 15K granulysin.

Specifically, treatment with a protein precipitating agent, ultrafiltration, gel filtration, high performance liquid chromatography, centrifugation, electrophoresis, affinity chromatography using a specific antibody, and dialysis method can be used alone or by combining these methods.

As described above, 15K granulysin can be separated and purified.

By using 15K granulysin as an active ingredient in a remedy for infectious diseases, it is incorporated into a macrophage in blood, and activated therein, whereby, it kills and injures bacteria, viruses, and fungi inducing infections which have been phagocytosed by a macrophage, whereby, it becomes possible to treat infections due to these microoraganisms. As described above, unlike 9K granulysin, cytotoxicity is not perceived in 15K granulysin per se, and effects as a remedy for infectious diseases having little side effect due to administration are expected.

(ii) Intracorporeal Expression Vector for 15K Granulysin.

An active ingredient of the present remedy in this form is a recombinant vector in which a gene encoding 15K granulysin used for expressing the aforementioned recombinant protein is inserted into an intracorporeal expression vector.

Examples of the intracorporeal expression vector are not limited to, but include an adenovirus vector and a retrovirus vector.

For a recombinant intracorporeal expression vector, for example, an expressible gene in which 15K granulysin is further transduced into a cosmid vector in which the aforementioned virus gene has been transduced, is incorporated and then, this cosmid vector and a parent virus DNA—TP which have been restriction enzyme treated are transfected into 293 cells, whereby, homologous recombination occurs in the 293 cells, thus, a desired intracorporeal expression vector is produced.

B. Form of Present Remedy

(i) Present Remedy Using Recombinant Protein of 15K Granulysin as Active Ingredient.

In the first form of the present remedy, 15K granulysin is incorporated as an active ingredient and, at the same time, an appropriate pharmaceutical preparation carrier can be incorporated to prepare into a form of a preparation composition (of course, only 15K glanulysin is possible). As a pharmaceutical preparation carrier, for example, excipients and diluents such as fillers, bulking agents, binders, wetting agents, stabilizers, solubilizers, disintegrating agents and surface active agents which can be conventionally used as a pharmaceutical preparation carrier can be appropriately selected freely depending on a specific dosage form. A form of a preparation composition is not particularly limited as far as it is such a form that 15K granulysin can be effectively used in utilities of treatment of infectious diseases. For example, even solid agents such as tablets, powders, granules and pills may be formulated into an injectable form such as solutions, suspensions and emulsions. By adding an appropriate carrier to 15K granulysin, a dried agent can be obtained which can be made to be liquid upon use.

A dose of the thus obtained present remedy can be appropriately selected depending on an administration method and an administration form of an agent, and symptom of a patient, being not particularly limited.

Such the various forms of pharmaceutical preparations can be administered by an appropriate administration route depending on its form, for example, by intravenous, intramuscular, intraosseous, intra-articular, subcutaneous, intracutaneous or intraperitoneal administration in the case of an injectable form, or by oral or enteral administration in the case of a solid agent form.

(ii) Present Remedy Using Intracorporeal Expression Vector for 15K Granulysin as an Active Ingredient.

By isolating and purifying the intracorporeal expression vector which can be prepared as described above, and administering this to a living body, 15K granulysin is produced in a living body, and the pharmacological effect of this 15K granulysin can be exerted.

A dosage form in this case is generally an injectable form, and the remedy can be administered by intravenous, intramuscular, intraosseous, intra-articular, subcutaneous, intracutaneous or intraperitoneal administration. A dose of such the present remedy can be appropriately selected depending on an administration method and an administration form of an agent, and symptom of a patient, being not limited. Generally, it is preferable to appropriately prepare an intracorporeal expression vector expressing 15K granulysin as an active ingredient, and administer this preparation at an appropriate amount once per day or by dividing into a few times per day.

### EXAMPLE

Examples of the present invention will be described below.

[Test Example] Study of Antibacterial effect on Mycobacterium tuberculosis when 15K Granulysin and a Macrophage are Present Jointly

(1) Preparation of Monoclonal Antibody Specific for 15K Granulysin

A RNA was extracted from human peripheral blood lymphocyte which had been cultured in the presence of 100 to 200 unit/ml of IL-2 (2×10₆ cells/ml of human peripheral blood lymphocytes were cultured at 37° C. under 5% CO₂ incubator for 10 days in RPMI1640 medium containing 10% fetal bovine serum) by the conventional method, and a RT-PCR method (PCR primer1: SEQ ID NO:2, PCR primer 2:SEQ ID NO: 3) was performed using this RNA as a template, to synthesize a gene part containing a region encoding a full length protein of 15K granulysin (Jongstra et al., J. Exp. Med, 165, 601: part corresponding to amino acid sequence of SEQ ID NO:1] as the amplification product of a complementary DNA (cDNA). This cDNA encoding a full length protein of 15K granulysin was incorporated into pRc/CMV or pcDL.SRa296 which is a mammal expression vector, the resulting recombinant vector was dissolved in a physiological saline, and a mouse was immunized with the solution subcutaneously or intracutaneously. After 4 to 5 times immunization at an interval of 1 to 2 weeks, spleen cells obtained from mouse for which increase in an antibody titer was perceived by an indirect fluorescent antibody method (performed according to the method described later) was cell-fused according to the conventional method, and a hybridoma producing an antibody which specifically binds to granulysin was retrieved again with an indirect fluorescent antibody method. That is, the cells were transfected with the aforementioned gene encoding 15K granulysin, cells (Cos7) in which the gene was expressed was fixed with 4% paraformaldehyde, membranes were solubilized with 0.5% Tween 20, the culture supernatant of hybridoma cells was added thereto to react with an antibody, this was reacted with a fluorescently labeled anti-mouse IgG antibody, and fluorescence was detected, whereby, a hybridoma producing an antibody which specifically binds to granulysin was screened. As a result, 9 hybridcmas producing an antibody which specifically binds to granulysin were obtained. Using the culture supernatant of each of the resulting hybridomas, ammonium sulfate precipitation and purification by Protein G column were performed to prepare two kinds of monoclonal antibodies to 15K granulysin. Hereinafter, this is referred to monoclonal antibody RF10 (which binds to 15K granulysin, but does not bind to 9K granulysin) or monoclonal antibody RC8 (which binds to both 15K granulysin and 9K granulysin).

(2) Preparation of polyclonal antibody to 15K granulysin. A rabbit was immunized with a conjugate of a partial amino acid sequence (29 amino acids) of granulysin (J. Exp. Med. 165:601-614 (1987), J. Exp. Med., 172:1159-1163 (1990)): Arg Thr Gly Arg Ser Arg Trp Arg Asp Val Cys Arg Aso Phe Met Arg Arg Tyr Gln Ser Arg Val Thr Gln Gly Leu Val Ala Gly (N5-1: SEQ ID NO: 4) and limpet hemocyanin according to the conventional method, to obtain anti-serum. The resulting antiserum was purified by ammonium sulfate precipitation and Protein G column, and further purified by affinity chromatography using a column bound with the aforementioned synthetic peptide (N5-1), to prepare a polyclonal antibody (anti-N5-1 antibody) to granulysin.

(3) Preparation of Granulysin Containing Culture Supernatant.

A gene recombinant vector was produced, in which, among a nucleotide sequence of SEQ ID NO:1, a nucleotide chain of a nucleotide sequence encoding a part corresponding to 15K granulysin protein was incorporated into pFLAG-CMV vector (manufactured by Sigma) according to the conventional method. As a control, the pFLAG-CMV vector in which recombination with a gene was not performed, was used. Each of these gene recombinant vectors was transfected into a Cos7 cell, and this was cultured at 37° C. under 5% CO₂ for 72 hours in DMEM medium (manufactured by Gibco). After completion of culture, the culture was centrifuged (2500 rpm·20 min·4° C.) to obtain the culture supernatant.

Regarding each of the culture supernatants, the proteins were separated by SDS-PAGE. After the protein was transferred to a nylon membrane from a gel of electrophoresed SDS-PAGE, the membrane was blocked with blocking solution (1% skim milk/washing solution), to this was bound monoclonal antibody RF10 specific for 15K granulysin, and enzyme-labeled anti-mouse antibody and color developing substrate were acted on these, to develop a bond. As a result, in the supernatant obtained by expressing a gene encoding 15K granulysin protein, a band exhibiting a molecular weight of 15K appeared. However, in the control, this bond was not perceived In addition, when the similar test was performed using a polyclonal antibody N5-1 binding to 15K and 9K granulysins, a band showing 15K was perceived, but a band showing 9K was not perceived.

From this result, it was revealed that 15K granulysin was specifically present in the aforementioned granulysin culture supernatant, but granulysin was not present in control culture supernatant.

(4) Study of Antibacterial Effect

Lymphocyte separated from human blood was suspended in a culture (RPMI1640, 10% human serum) to around 2×107 cells in a plastic culture plate (24 wells/plate) per 1 ml of medium according to the conventional method, each 1 ml of this was dispensed into each well, allowed to stand at 37° C. for 24 hours, lymphocyte was adhered to a plate surface wall and, in this adherent macrophage, antibacterial effect of 15K granulysin was studied.

Then, 1 ml of RFMI1640 (10% human serum) was added to a well, Mycobacterium tuberculosis (M. tuberculosis) (H37Rv, 1×10⁵ to 1×10⁶cfu) was static-cultured at 37° C. for 4 to 12 hours under 5% CO₂, to infect macrophages with M. tuberculosis. After completion of infection, each 1 ml of supernatant containing 15K granulysin (Grn supernatant) and supernatant in the absence of 15K granulysin (Cont supernatant) was added to a well, and static culture was performed 2 to 12 hours under the same conditions.

After completion of culture, the culturing supernatant was removed, the adhered cells in a plate were washed with PBS three times, M. tuberculosis in a cell was extracted with total 5 ml of 1% aqueous saponin solution, this extract was diluted, seeded on a plate agar medium [7H1 1 medium (manufactured by Gibco)], this was static-cultured at 37° C. for 14 days, and the number of colonies was counted.

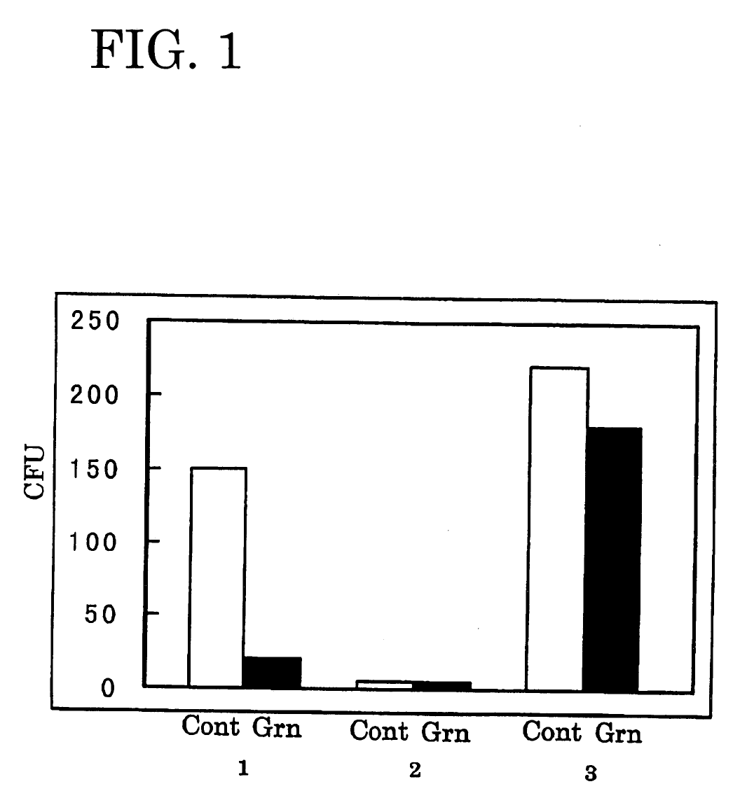

The results are shown in FIG. 1.

In FIG. 1, Cont denotes control culturing supernatant, and Grn denotes granulysin culture supernatant. A coordinate axis denotes the number of colonies. 1 on an abscissa axis denotes the results obtained by contacting macrophages, M. tuberculosis and the culture supernatant, as described above. 2 denotes the result obtained by static-culturing each culture supernatant and M. tuberculosis in the absence of macrophages as in 1, washing this, static-culturing this in a 7H11 plate agar medium, and influence of M. tuberculosis on non-specific adsorption in a plate was confirmed. 3 denotes the result obtained by static culture each 1 ml of the culture supernatant and M. tuberculosis at 37° C. for 2 hours in the aforementioned culture plate, and static-culture this in a 7H11 plate agar medium.

As a result, it was revealed that 15K granulysin has antibacterial effect on M. tuberculosis by intervention of macrophages. When macrophages did not intervene, antibacterial effect of 15K granulysin on M. tuberculosis was not perceived.

By the aforementioned Test Example, antibacterial activity was perceived in 15K granulysin, and it was revealed that 15K granulysin can be used as an active ingredient of a remedy for infections.

It has been reported that, when 15K granulysin was transfected into cells such as Cos7 cells, HeLa and PC12, 15K granulysin was detected in the culture supernatant, but damage was not perceived in the cells. When 9K granulysin was used in place of 15K granulysin in the presence of perforin, lo cytotoxic activity or apoptosis is induced (Pena, S. V. et al, J. Immunol., 158, 2680-2688(1997)).

1. A remedy for infectious diseases, which comprises 15K granulysin as an active ingredient.

2. The remedy for infectious diseases according to claim 1, wherein 15K granulysin is a recombinant protein.

3. A remedy for infectious diseases, which comprises, as an active ingredient, an intracorporeal expression vector for 15K granulysin, in which
a gene encoding 15K granulysin is incorporated
